(19) United States
(12) Patent Application Publication
(10) Pub. No.: US 2013/0247915 A1
(43) Pub. Date: Sep. 26, 2013

(54) PATIENT INTERFACE DEVICE WITH MULTI-AXIS ELBOW CONDUIT

(71) Applicant: Richard Thomas Haibach, Eindhoven (NL)

(72) Inventor: Richard Thomas Haibach, Eindhoven (NL)

(73) Assignee: KONINKLIJKE PHILIPS ELECTRONICS N.V., EINDHOVEN (NL)

(21) Appl. No.: 13/989,208

(22) PCT Filed: Nov. 21, 2011
(86) PCT No.: PCT/IB11/55203

§ 371 Date: May 23, 2013

Related U.S. Application Data
(60) Provisional application No. 61/418,903, filed on Dec. 2, 2010.

(51) Int. Cl. A61M 16/08

(52) U.S. Cl. CPC A61M 16/0816
USPC 128/206.21

(57) ABSTRACT

A patient interface device (2) includes a frame (14), a cushion (12) coupled to the frame, and a fluid coupling conduit (10) having a first conduit member (24) and a second conduit member (26), the first conduit member being fluidly coupled to at least one of the frame and the cushion and being rotatable with respect to the frame and about a longitudinal axis of the first conduit member, the second conduit member being fluidly coupled to first conduit member and being rotatable with respect to the first conduit member and about an axis that is transverse to the longitudinal axis of the first conduit member.

This patent application claims the priority benefit under 35 U.S.C. §119(e) of U.S. Provisional Application No. 61/418,903 filed on Dec. 2, 2010, the contents of which are herein incorporated by reference.

### BACKGROUND OF THE INVENTION

1. Field of the Invention

The present invention relates to respiratory patient interface devices, and, in particular, to a respiratory patient interface device having an elbow conduit that provides for movement in multiple axes.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Patients that must utilize the respiratory therapies as just described are often confronted with the problem of managing the tubing that extends between the ventilator or pressure support device and the patient interface device and carries the flow of gas from the ventilator or pressure support device to the patient interface device. In particular, they frequently struggle with tubing torque on the patient interface device as body movement occurs while asleep. Such tubing torque can negatively impact mask stability and result in a loss of the seal of the mask on the face of the patient. These issues often frustrate the patient and can interfere with the effective delivery of therapy.

### SUMMARY OF THE INVENTION

Accordingly, it is an object of the present invention to provide a patient interface device that overcomes the shortcomings of conventional patient interface device. This object is achieved according to one embodiment of the present invention by providing a patient interface device that includes a frame, a cushion coupled to the frame, and a fluid coupling conduit having a first conduit member and a second conduit member. The first conduit member is fluidly coupled to at least one of the frame and the cushion and rotatable with respect to the frame and about a longitudinal axis of the first conduit member. The second conduit member is fluidly coupled to first conduit member and is rotatable with respect to the first conduit member and about an axis that is transverse to the longitudinal axis of the first conduit member.

In another embodiment, a fluid coupling conduit for a patient interface device is provided that includes a first conduit member structured to be fluidly coupled to at least one of a frame and a cushion of the patient interface device and being rotatable with respect to the frame and about a longitudinal axis of the first conduit member when coupled to the frame, and a second conduit member, the second conduit member being fluidly coupled to first conduit member and being rotatable with respect to the first conduit member and about an axis that is transverse to the longitudinal axis of the first conduit member.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the present invention;

FIG. 2 is a side isometric view of an elbow conduit of a patient interface device forming a part of the system of FIG. 1 according to an exemplary embodiment of the invention;

FIG. 3 is a rear elevational view of the elbow conduit of FIG. 2;

FIG. 4 is a bottom isometric view of a first conduit member of the elbow conduit of FIGS. 2 and 3;

FIG. 5 is a side isometric view of the first conduit member of FIG. 4;

FIG. 6 is a bottom isometric view of a second conduit member of the elbow conduit of FIGS. 2 and 3;

FIG. 7 is a side isometric view of the second conduit member of FIG. 6;

FIG. 8 is an isometric view of an elbow conduit having a split ring washer attached thereto according to an alternative exemplary embodiment of the invention;

FIG. 9 is a top plan view of the split ring washer shown in FIG. 8 for securing the elbow conduit of FIG. 8 to a frame or faceplate of a patient interface device;

FIG. 10 is an isometric view of a swivel connector assembly forming part of the elbow conduit of FIG. 8;

FIGS. 11 and 12 are isometric views of first and second members, respectively, of the swivel connector assembly of FIG. 10;

FIG. 13 is a side elevational view of a pivot assembly forming part of the elbow conduit of FIG. 8;

FIG. 14 is a front elevational view of the pivot assembly of FIG. 13;

FIGS. 15-17 are a front isometric view, a front elevational view, and a bottom isometric view, respectively, of a first conduit member of the pivot assembly of FIGS. 13 and 14;

FIGS. 18 and 19 are a side elevational view and a front isometric view, respectively, of a second conduit member of the pivot assembly of FIGS. 13 and 14;

FIGS. 20 and 21 are side and isometric views of an elbow conduit according to another alternative exemplary embodiment of the invention;

FIG. 22 is an isometric view of a first conduit member of the elbow conduit of FIGS. 20 and 21; and

FIG. 23 is an isometric view of a second conduit member of the elbow conduit of FIGS. 20 and 21.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

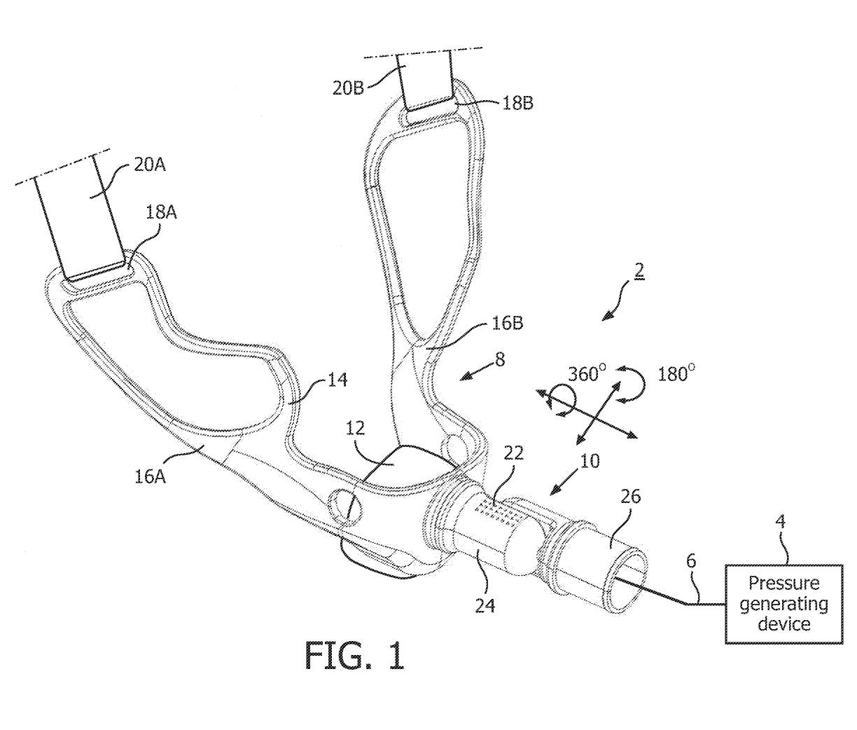

A system 2 adapted to provide a regimen of respiratory therapy to a patient according to one exemplary embodiment is generally shown in FIG. 1. System 2 includes a pressure generating device 4, a patient circuit 6, a patient interface device 8, and an elbow conduit 10 according to one exemplary embodiment of the present invention which is described in greater detail herein. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Patient circuit 6 is structured to communicate the flow of breathing gas from pressure generating device 4 to patient interface device 8, and typically includes a gas delivery conduit or tube coupled to elbow conduit 10.

In the illustrated embodiment, patient interface 8 is a nasal mask structured to be placed over the nose of a patient. However, the present invention contemplates that the patient interface device can be any device suited to communicate a flow of gas with an airway of patient, such as a nasal cushion having nasal prongs that are received within the patient's nares, a nasal/oral mask that covers the nose and mouth, or a full face mask that covers the patient's face. That is any device that facilitates the delivery of the flow of breathing gas to, and the removal of a flow of exhalation gas from, the airway of such a patient may be used as patient interface 8 while remaining within the scope of the present invention

In the embodiment shown in FIG. 1, patient interface 8 includes a cushion 12 that is coupled to a rigid or semi-rigid frame or faceplate 14. In the exemplary embodiment, frame 14 includes arms 16A and 16B each having a respective loop member 18A, 18B for receiving a respective strap 20A, 20B of a headgear component to secure patient interface device 8 to the patient's head. An opening in frame 14 to which elbow conduit 10 is coupled allows the flow of breathing gas from pressure generating device 4 to be communicated to an interior space defined by cushion 12, and then, to the airway of a patient. The opening in frame 14 also allows the flow of exhalation gas (from the airway of such a patient) to be communicated to an exhaust port 22 of elbow conduit 10 in the current embodiment. It will be appreciated that exhaust port need not be provided as part of elbow conduit 10, but rather may be provided in a portion of patient interface device 8, such as part of frame 14.

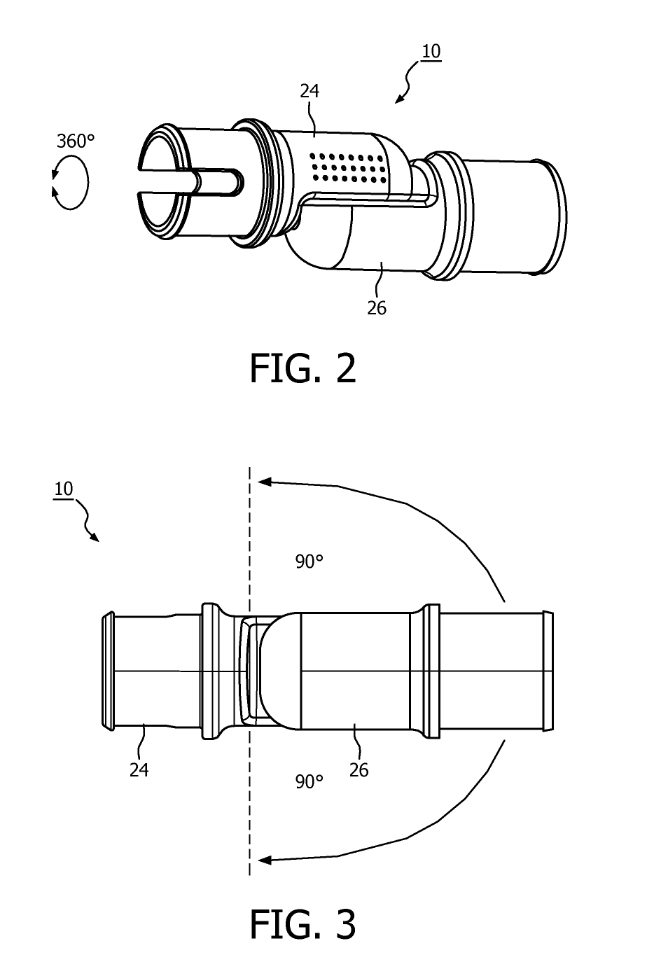

FIG. 2 is a side isometric view and FIG. 3 is a rear elevational view of elbow conduit 10 according to an exemplary embodiment of the invention. As described above, elbow conduit 10 is structured to be coupled to patient interface device 8 by being inserted into or otherwise coupled to an opening provided in frame 14, and is also structured to be coupled to patient circuit 6, which is a flexible tube or conduit, so that a breathing gas can be delivered from pressure generating device 4 to patient interface device 8. In addition, as described in greater detail herein, elbow conduit 10 is structured to provide for simultaneous rotation in multiple axes with respect to frame 14.

Elbow conduit 10 includes a first conduit member 24 and a second conduit member 26 that are rotatably coupled to one another as described herein. As seen in FIG. 1, first conduit member 24 is the part of elbow conduit 10 that provides the coupling to frame 14, and second conduit member 26 is the part of elbow conduit 10 that provides the coupling to patient circuit 6.

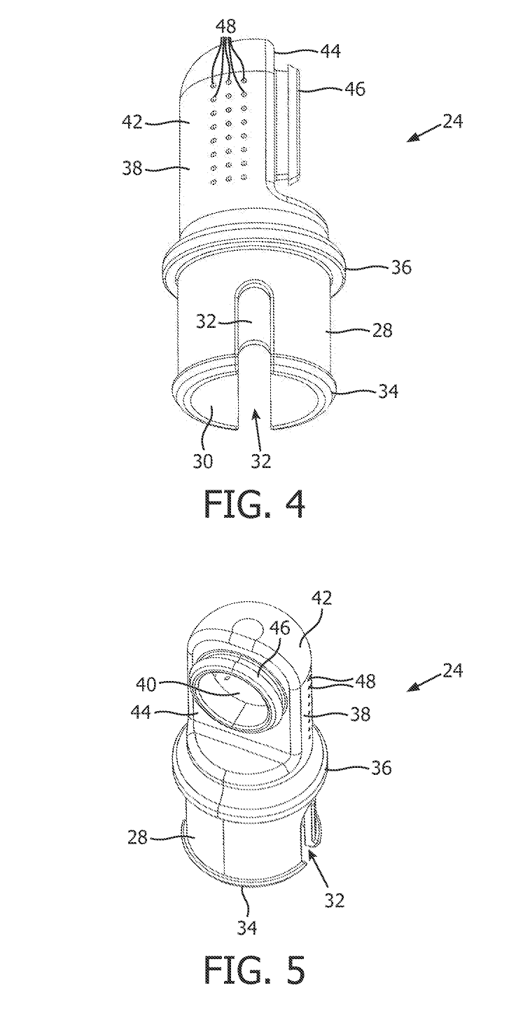

FIG. 4 is a bottom isometric view and FIG. 5 is a side isometric view of first conduit member 24. First conduit member 24 includes cylindrical connector portion 28 defining a first fluid chamber 30. Cylindrical connector portion 28 is provided with U-shaped cut-out portions 32, the function of which is described elsewhere herein. A bottom lip 34 is provided at the bottom of cylindrical connector portion 28, and a top lip 36 is provided at the top of cylindrical connector portion 28. A semi-cylindrical mating portion 38 is coupled to cylindrical connector portion 28 and defines a second fluid chamber 40 (FIG. 5) that is fluidly coupled to first fluid chamber 30. Semi-cylindrical mating portion 38 includes an arced surface 42 connected to a flat surface 44. A port member 46 extends from flat surface 44 and provides fluid access to second fluid chamber 40. In the exemplary embodiment, a plurality of apertures 48 are provided on arced surface for forming exhaust port 22 described elsewhere herein. First conduit member 24 thus defines a fluid path that extends from cylindrical connector portion 28 to semi-cylindrical mating portion 38 to port member 46.

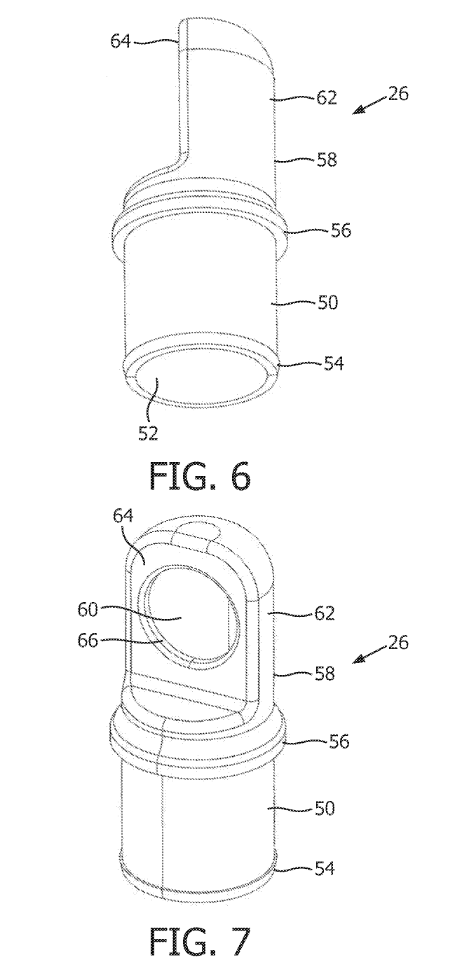

FIG. 6 is a bottom isometric view and FIG. 7 is a side isometric view of second conduit member 26. Second conduit member 26 includes cylindrical connector portion 50 defining first fluid chamber 52. A bottom lip 54 is provided at the bottom of cylindrical connector portion 50, and a top lip 56 is provided at the top of cylindrical connector portion 50. A semi-cylindrical mating portion 58 is coupled to cylindrical connector portion 50 and defines a second fluid chamber 50 (FIG. 7) that is fluidly coupled to first fluid chamber 52. Semi-cylindrical mating portion 58 includes an arced surface 62 connected to a flat surface 64. An orifice 66 is provided in flat surface 64 and provides fluid access to second fluid chamber 60. Second conduit member 26 thus defines a fluid path that extends from cylindrical connector portion 50 to semi-cylindrical mating portion 58 to orifice 66. In one particular, non-limiting exemplary embodiment, second conduit member 26 may further include an entrainment valve.

Elbow conduit 10 is assembled by inserting port member 46 of first conduit member 24 into orifice 66 of second conduit member 26. When this is done, flat surfaces 44, 64 will engage one another (FIG. 2), and second conduit member 26 will, in the exemplary embodiment, be able to rotate relative to first conduit member 24 over at least 180 degrees (FIG. 3). More specifically, second conduit member 26 will be able to rotate about longitudinal axes through port member 46 and orifice 66, which longitudinal axes are perpendicular to the longitudinal axis of both cylindrical connector portion 28 and cylindrical connector portion 50. In alternative exemplary embodiments, second conduit member 26 will be able to rotate relative to first conduit member 24 over less than 180 degrees, over 10-180 degrees, over 20-180 degrees or over 0-200 degrees.

Alternatively, port member 46 may be replaced by an orifice and orifice 66 may be replaced by a port member such that the port member of second conduit member 26 is inserted into the orifice of first conduit member 24 during assembly. In either exemplary embodiment, the port member is held within the orifice by a snap fit. In one particular exemplary embodiment, the port member may be provided with slots (similar to U-shaped cut-out portions 32 described below) to facilitate the snap fit insertion into the orifice.

Once assembled as just described, cylindrical connector portion 28 of first conduit member 24 may be inserted into the opening in frame 14. In the exemplary embodiment, U-shaped cut-out portions 32 allow cylindrical connector portion 28 to be temporarily compressed and reduced in diameter so that it can be inserted into the opening in frame 14 where it can snap in place after the compression force in removed. Once so connected, elbow conduit 10 is free to rotate within frame 14 about the longitudinal axis of first conduit member 24. In addition, after elbow conduit 10 is connected to frame 14 as just described, the delivery conduit or tube of patient circuit 6 can be connected to cylindrical connector portion 50 of second conduit member 26. In the exemplary embodiment, cylindrical connector portion 50 is sized to fit either a standard 15 mm hose or a standard 22 mm hose. In addition, a swivel connector may or may not be used for this connection.

Thus, when fully assembled as shown in FIG. 1, elbow conduit 10, and, thus, the delivery conduit or tube of patient circuit 6 coupled thereto, is able to rotate 360 degrees about the longitudinal axis of first conduit member 24. In addition, second conduit member 26, and thus the delivery conduit or tube of patient circuit 6 coupled thereto, is able to simultaneously rotate at least 180 degrees about an axis that is perpendicular to the longitudinal axis of first conduit member 24. This simultaneous, multi-axis rotation capability facilitates the movement of the tubing of patient circuit 6, and as a result will reduce the adverse effects of tubing torque, resulting in increased mask stability and a decreased likelihood of seal loss.

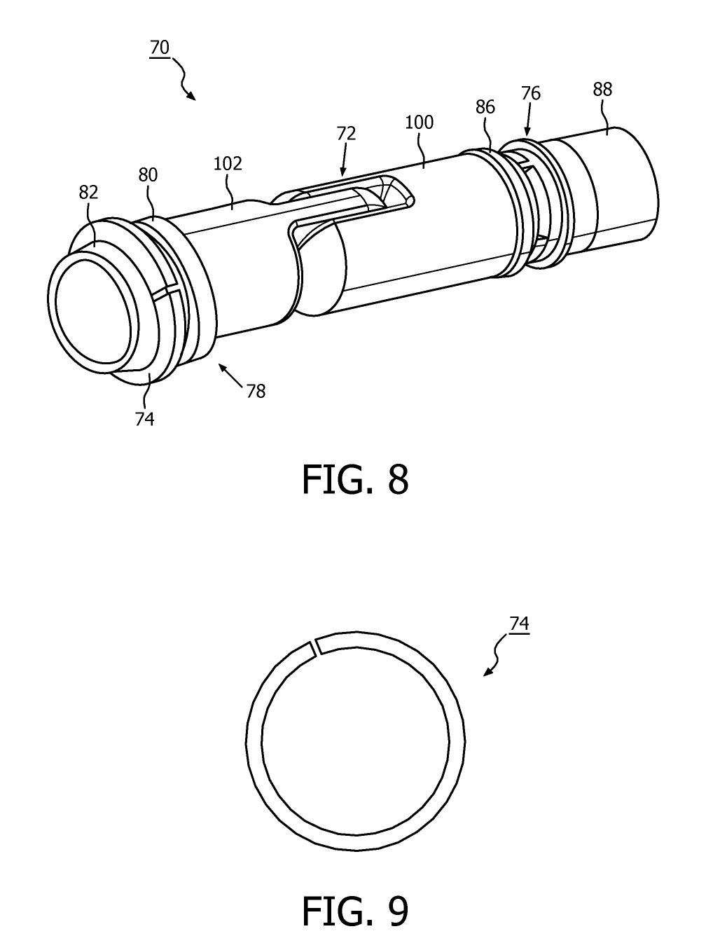

FIG. 8 is an isometric view of an elbow conduit 70 according to an alternative exemplary embodiment of the invention. Elbow conduit 70 may be coupled to patient interface device 8 by being inserted into an opening provided in frame 14 (or an alternative patient interface device by being inserted into an opening provided in the frame or faceplate thereof), and may coupled to a gas delivery conduit or tube of patient circuit 6 so that a breathing gas can be delivered from pressure generating device 4 to patient interface device 8. In addition, as described in greater detail herein, elbow conduit 70, like elbow conduit 10, is structured to provide for simultaneous rotation in multiple axes with respect to frame 14. Elbow conduit 70 includes a pivot assembly 72 and a swivel connector assembly 76, each of which is described in greater detail herein. In addition, as also described in greater detail herein, a split ring washer 74 is provided to secure elbow conduit 70 to frame 14. FIG. 9 is a top plan view of split ring washer 74.

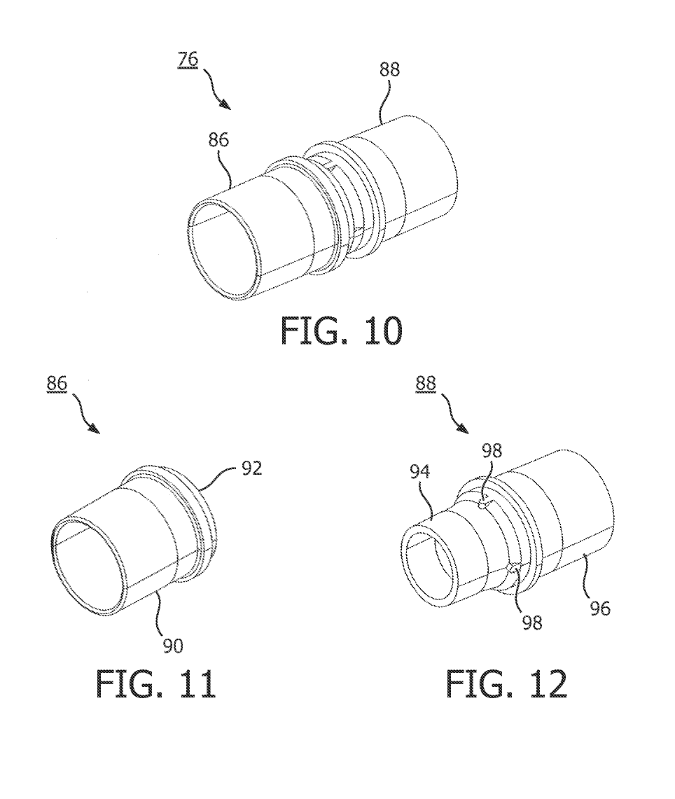

FIG. 10 is an isometric view of swivel connector assembly 76. As seen in FIG. 10, swivel connector assembly 76 includes a first member 86 and a second member 88 rotatably coupled to one another. FIG. 11 is an isometric view of first member 86. First member 86 includes a cylindrical body portion 90 that is structured to be coupled to pivot assembly 72, and a flat, circular engagement surface 92 located at one end cylindrical body portion 90. FIG. 12 is an isometric view of second member 88. Second member 88 includes a first cylindrical body portion 94 structured to be received within first member 86 in a manner that allows for relative rotation between the two components, and a larger diameter second cylindrical body portion 96 provided adjacent first cylindrical body portion 94. In addition, a plurality of individual, arc-shaped engagement structures 98 are provided along and spaced apart from one another about an outer peripheral portion of the end second cylindrical body portion 96 closest to first cylindrical body portion 94.

Swivel connector assembly 76 is assembled by inserting first cylindrical body portion 94 into first member 86. When this is done, engagement structures 98 will engage engagement surface 92 create a plurality of point-contacts between first member 86 and second member 88. The creation of point contacts instead of a complete face to face or line contact by the plurality of engagement surfaces reduces or minimizes friction between first member 86 and second member 88, thereby facilitating the relative rotation between the two components.

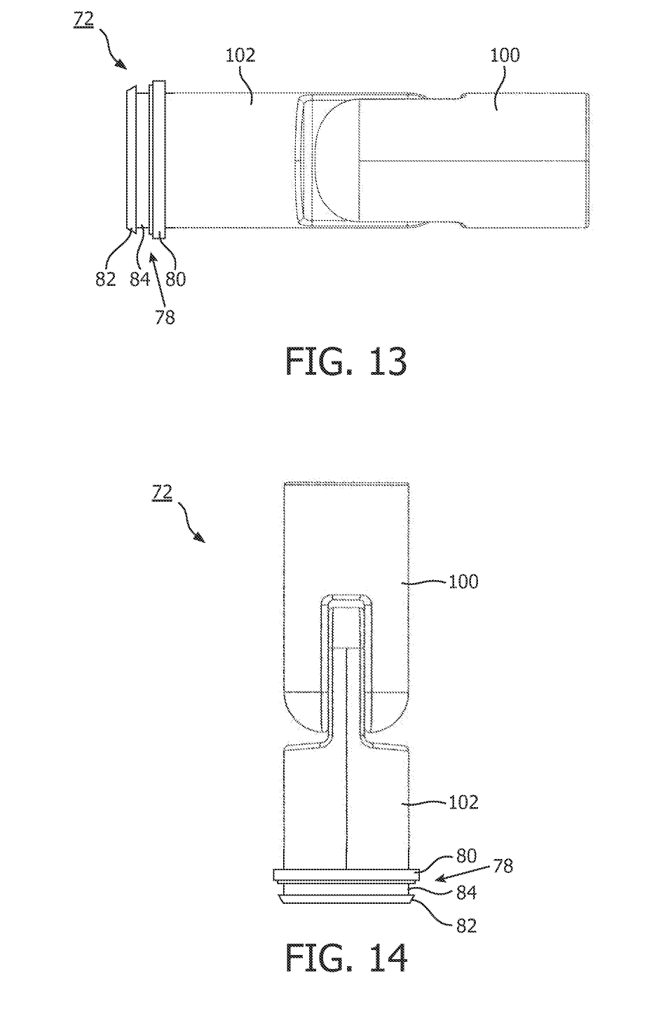

FIG. 13 is a side elevational view and FIG. 14 is a front elevational view of pivot assembly 72. As seen in FIGS. 13 and 14, pivot assembly 72 includes first conduit member 100 and second conduit member 102 that are rotatably coupled to one another as described herein.

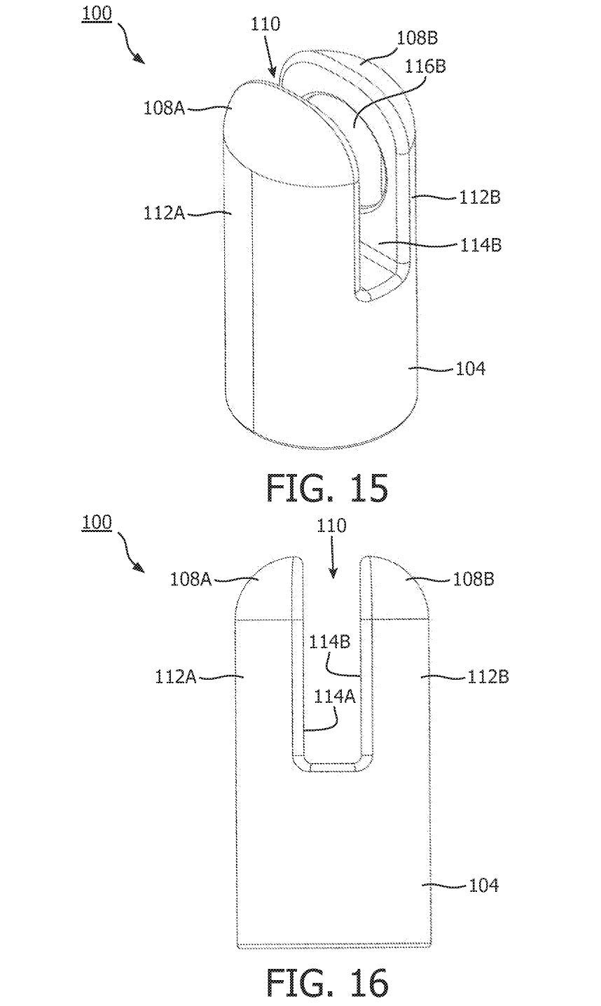

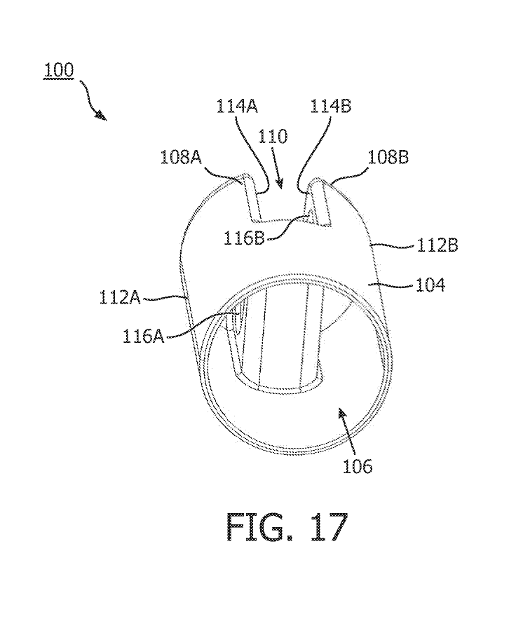

FIG. 15 is a front isometric view, FIG. 16 is a front elevational view, and FIG. 17 is a bottom isometric view of first conduit member 100. First conduit member 100 includes cylindrical connector portion 104 defining first fluid chamber 106 (FIG. 17). First and second semi-cylindrical mating portions 108A, 108B are coupled to cylindrical connector portion 104 and define a gap 110 between them. In addition, each semi-cylindrical mating portion 108A, 108B defines an internal fluid chamber that is fluidly coupled to first fluid chamber 106. Also, each semi-cylindrical mating portion 108A, 108B includes an arced surface 112A, 112B connected to a flat surface 114A, 114B. An orifice 116A, 116B is provided in each flat surface 114A, 114B and provides fluid access to each internal fluid chamber. First conduit member 100 thus defines a fluid path that extends from cylindrical connector portion 104 to semi-cylindrical mating portions 108A, 108B to orifices 116A, 116B. In an alternative embodiment, one of first and second semi-cylindrical mating portions 108A, 108B may be solid and therefore not include an internal fluid chamber that is fluidly coupled to first fluid chamber 106. In this embodiment, first conduit member 100 will defines a fluid path that extends from cylindrical connector portion 104 to the non-solid semi-cylindrical mating portions 108A, 108B to associated orifices 116A, 116B.

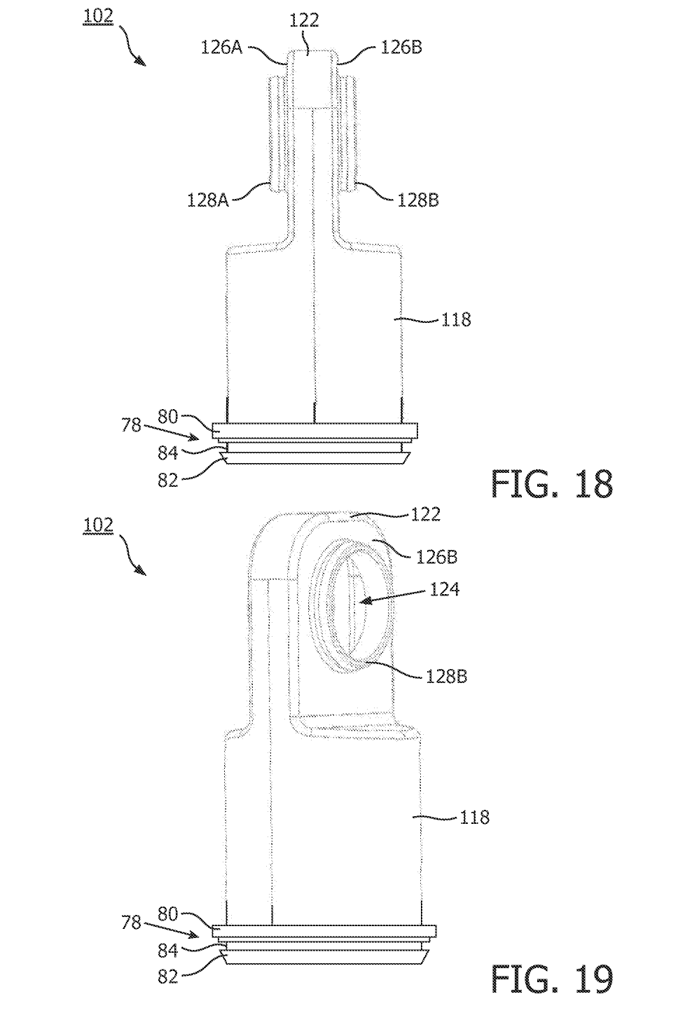

FIG. 18 is a side elevational view and FIG. 19 is a front isometric view of second conduit member 102. Second conduit member 102 includes cylindrical connector portion 118 defining first fluid chamber 120. In addition, cylindrical connector portion 118 includes a snap ring connector portion 78 for securing elbow conduit 70, once assembled as described herein, to frame 14 or another frame or faceplate. More specifically, snap ring connector portion 78 includes rings 80, 82 which define a groove 84 therebetween. Groove 84 is structured to receive split ring washer 74 (FIG. 9) after snap ring connector portion 78 is inserted through the opening provided in frame 14 (or a similar opening in another frame or faceplate) to keep snap ring connector portion 78 from pulling back through the opening.

A mating portion 122 is coupled to cylindrical connector portion 118 and defines a second fluid chamber 124 (FIG. 19) that is fluidly coupled to first fluid chamber 120. Mating portion 122 includes first and second flat surfaces 126A, 126B positioned opposite one another. A port member 128A, 128B extends from each flat surface 126A, 126B and provides fluid access to second fluid chamber 124. Second conduit member 102 thus defines a fluid path that extends from cylindrical connector portion 118 to mating portion 122 to port members 128A, 128B. In an alternative exemplary embodiment, one of port members 128A, 128B may be closed off with a plug or the like, in which case second conduit member 102 will define a fluid path that extends from cylindrical connector portion 118 to mating portion 122 to the open one of the port members 128A, 128B.

Pivot assembly 72 is assembled by inserting mating portion 122 of second conduit member 102 into gap 110 of first conduit member 100 and inserting port members 128A, 128B into orifices 116A, 116B. When this is done, flat surfaces 126A, 126B will engage flat surfaces 114A, 114B, and first conduit member 100 will be able to rotate relative to second conduit member 102 over at least 180 degrees. More specifically, first conduit member 100 will be able to rotate about longitudinal axes through 128A, 128B, which longitudinal axes are perpendicular to the longitudinal axis of both cylindrical connector portion 104 and cylindrical connector portion 118.

Furthermore, the remainder of elbow conduit 70 may be assembled as shown in FIG. 8 by inserting first member 86 of swivel connector assembly 76 into cylindrical connector portion 104 of first conduit member 100 of pivot assembly 72. Snap ring connector portion 78 may then be inserted into the opening in frame 14. Once so connected, elbow conduit 70 is free to rotate within frame 14 about the longitudinal axis of second conduit member 102. In addition, after elbow conduit 70 is connected to frame 14 as just described, the delivery conduit or tube of patient circuit 6 can be connected to swivel connector assembly 76.

Thus, when fully assembled, elbow conduit 70, and thus the delivery conduit or tube of patient circuit 6 coupled thereto, is able to rotate 360 degrees about the longitudinal axis of second conduit member 102. In addition, first conduit member 100, and thus the delivery conduit or tube of patient circuit 6 coupled thereto, is able to simultaneously rotate at least 180 degrees about an axis that is perpendicular to the longitudinal axis of second conduit member 102. This simultaneous, multi-axis rotation capability facilitates the movement of the tubing of patient circuit 6, and as a result will reduce the adverse effects of tubing torque, resulting in increased mask stability and a decreased likelihood of seal loss.

Alternatively, the first conduit member 100 side of pivot assembly 72 could be attached to frame 14 and the second conduit member 102 side of pivot assembly 72 could be attached to patient circuit 106 with the same functionality being provided.

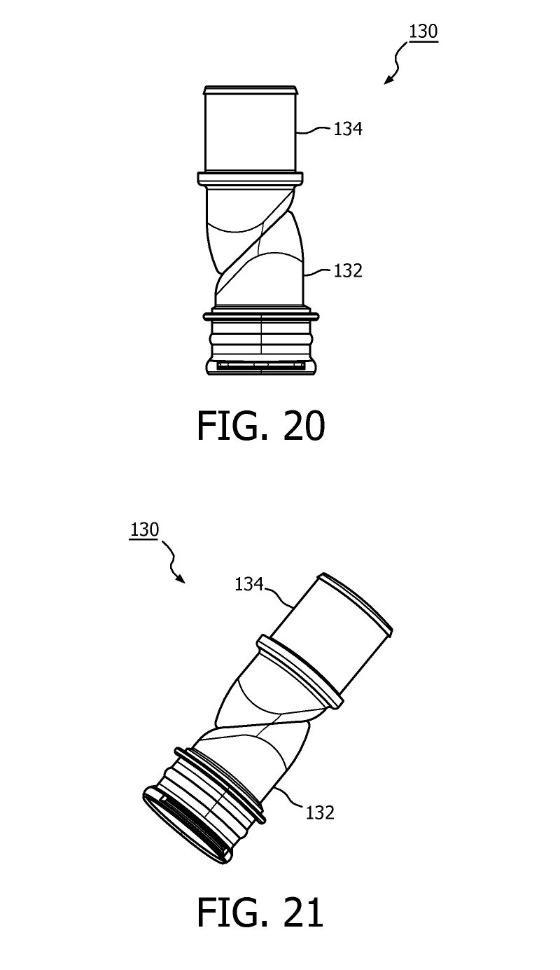

FIG. 20 is a side view and FIG. 21 is an isometric view of an elbow conduit 130 according to another alternative exemplary embodiment of the invention. Elbow conduit 130 may be coupled to patient interface device 8 by being inserted into an opening provided in frame 14 (or an alternative patient interface device by being inserted into an opening provided in the frame or faceplate thereof), and may coupled to a gas delivery conduit or tube of patient circuit 6 so that a breathing gas can be delivered from pressure generating device 4 to patient interface device 8. In addition, as described in greater detail herein, elbow conduit 130, like elbow conduits 10 and 70, is structured to provide for simultaneous rotation in multiple axes with respect to frame 14.

Elbow conduit 130 includes a first conduit member 132 and a second conduit member 134 that are rotatably coupled to one another as described herein. First conduit member 132 is the part of elbow conduit 130 that provides the coupling to frame 14, and second conduit member 134 is the part of elbow conduit 10 that provides the coupling to patient circuit 6.

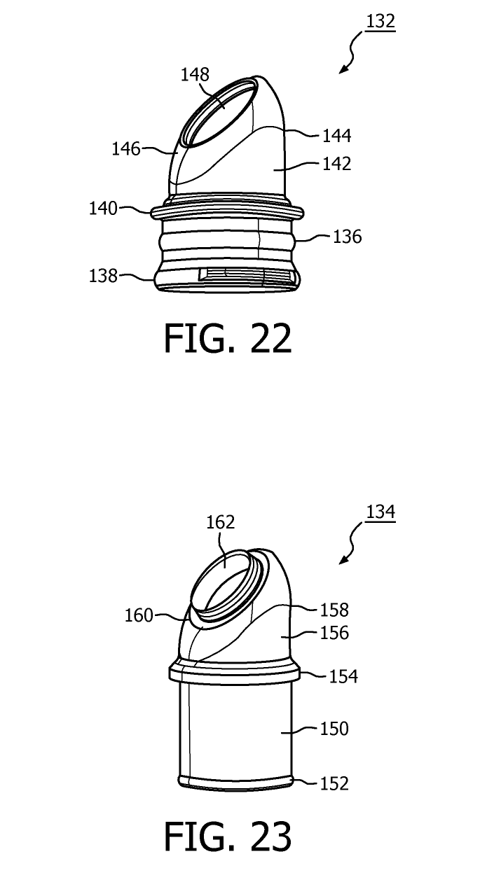

FIG. 22 is an isometric view of first conduit member 132, which is a female type connector. First conduit member 132 includes cylindrical connector portion 136 defining a first fluid chamber. A bottom lip 138 is provided at the bottom of cylindrical connector portion 136, and a top lip 140 is provided at the top of cylindrical connector portion 136. An angled mating portion 142 is coupled to cylindrical connector portion 136 and defines a second fluid chamber that is fluidly coupled to the first fluid chamber of first conduit member 132. Mating portion 142 includes a back surface 144 connected to an angled surface 146. Angled surface 146 is disposed at an angle with respect to the flat plane defining the top of cylindrical connector portion 136. In the exemplary embodiment, that angle may range from 30 degrees to 90 degrees (at 90 degrees, it will be similar to that shown in the embodiment of FIGS. 1-7). In the illustrated embodiment, the angle at which angled surface 146 is disposed is 45 degrees. The significance of this is described elsewhere herein. An orifice 148 is provided in angled surface 146 and provides fluid access to the second fluid chamber of first conduit member 132. A plurality of apertures (not shown) may be provided in back surface 144 for forming an exhaust port. First conduit member 132 thus defines a fluid path that extends from cylindrical connector portion 136 to angled mating portion 142 to orifice 148.

FIG. 23 is an isometric view of second conduit member 134, which is a male type connector. Second conduit member 134 includes a cylindrical connector portion 150 defining a first fluid chamber. A bottom lip 152 is provided at the bottom of cylindrical connector portion 150, and a top lip 154 is provided at the top of cylindrical connector portion 150. An angled mating portion 156 is coupled to cylindrical connector portion 150 and defines a second fluid chamber that is fluidly coupled to the first fluid chamber. Mating portion 156 includes a back surface 158 connected to an angled surface 160. Angled surface 160 is disposed at an angle with respect to the flat plane defining the top of cylindrical connector portion 150 that matches the angle of angled surface 146 of first conduit member 132. In the exemplary embodiment, that angle may range from 30 degrees to 90 degrees (at 90 degrees, it will be similar to that shown in the embodiment of FIGS. 1-7). In the illustrated embodiment, the angle at which angled surface 160 is disposed is 45 degrees. The significance of this is described elsewhere herein. A male port member 162 extends from angled surface 160 and provides fluid access to the second fluid chamber of second conduit member 134. Second conduit member 134 thus defines a fluid path that extends from cylindrical connector portion 150 to mating portion 156 to port member 162. In one particular, non-limiting exemplary embodiment, second conduit member 134 may further include an entrainment valve.

Elbow conduit 130 is assembled by inserting port member 162 of second conduit member 134 into orifice 148 of first conduit member 132. When this is done, angled surfaces 146, 160 will engage one another (FIGS. 20 and 21), and second conduit member 134 will, in the exemplary embodiment, be able to rotate relative to first conduit member 132 over 360 degrees. More specifically, second conduit member 134 will be able to rotate about longitudinal axes through port member 162 and orifice 148.

Alternatively, port member 162 may be replaced by an orifice and orifice 148 may be replaced by a port member such that the port member of first conduit member 132 is inserted into the orifice of second conduit member 134 during assembly. In either exemplary embodiment, the port member is held within the orifice by a snap fit. In one particular exemplary embodiment, the port member may be provided with slots to facilitate the snap fit insertion into the orifice.

Once assembled as just described, cylindrical connector portion 136 of first conduit member 132 may be inserted into the opening in frame 14. Once so connected, elbow conduit 130 is free to rotate within frame 14 about the longitudinal axis of first conduit member 132. In addition, after elbow conduit 130 is connected to frame 14 as just described, the delivery conduit or tube of patient circuit 6 can be connected to cylindrical connector portion 150 of second conduit member 134. In the exemplary embodiment, cylindrical connector portion 150 is sized to fit either a standard 15 mm hose or a standard 22 mm hose. In addition, a swivel connector may or may not be used for this connection.

Thus, when fully assembled as shown in FIGS. 20 and 21, elbow conduit 130, and, thus, the delivery conduit or tube of patient circuit 6 coupled thereto, is able to rotate 360 degrees about the longitudinal axis of first conduit member 132. In addition, second conduit member 134, and thus the delivery conduit or tube of patient circuit 6 coupled thereto, is able to simultaneously rotate about an axis that is transverse to the longitudinal axis of first conduit member 132. In particular, second conduit member 134 is able to rotate from a first position wherein the longitudinal axis of second conduit member 134 is aligned with and parallel to the longitudinal axis of second conduit member 134 as seen in FIG. 20 and 21, to a second position wherein the longitudinal axis of second conduit member 134 perpendicular to the longitudinal axis of second conduit member 134. This simultaneous, multi-axis rotation capability facilitates the movement of the tubing of patient circuit 6, and as a result will reduce the adverse effects of tubing torque, resulting in increased mask stability and a decreased likelihood of seal loss.

It can be appreciated that the present invention facilitates movement of the tubing between the ventilator or pressure support device and the patient interface device in order to reduce the effects of tubing torque to increase mask stability and decrease the likelihood of loss of seal.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a frame;
a cushion coupled to the frame; and
a fluid coupling conduit having a first conduit member and a second conduit member, the first conduit member being fluidly coupled to at least one of the frame and the cushion and being rotatable with respect to the frame and about a longitudinal axis of the first conduit member, the second conduit member being fluidly coupled to first conduit member and being rotatable with respect to the first conduit member and about an axis that is transverse to the longitudinal axis of the first conduit member,
wherein the first conduit member comprises a first connector portion defining a first fluid chamber, and a first mating portion coupled to the first connector portion, the first mating portion defining a second fluid chamber,
wherein the second conduit member comprises a second connector portion defining a third fluid chamber, and a second mating portion coupled to the second connector portion, the second mating portion defining a fourth fluid chamber,
wherein the first mating portion is fluidly coupled to the second mating portion,
wherein the first mating portion includes a first back surface connected to a first angled surface, the first angled surface being disposed at a first angle with respect to a plane defining a flat top of the first connector portion,
wherein the second mating portion includes a second back surface connected to a second angled surface, the second angled surface being disposed at a second angle with respect to a plane defining a flat top of the second connector portion, and wherein the first angle is the same as the second angle, and
wherein the first angle and the second angle are each less than 90 degrees and greater than 30 degrees.

2. (canceled)

3. The patient interface device according to claim 1, wherein the first conduit member is rotatable with respect to the frame and about the longitudinal axis of the first conduit member over 360 degrees.

4. The patient interface device according to claim 1, wherein the second conduit member is rotatable with respect to the first conduit member and about the axis over at least 180 degrees.

5. The patient interface device according to claim 3, wherein the second conduit member is rotatable with respect to the first conduit member and about the axis over greater than 180 degrees.

6. The patient interface device according to claim 1, wherein the second conduit member is rotatable with respect to the first conduit member and about the axis over less than 180 degrees.

7. The patient interface device according to claim 1, further comprising a gas delivery conduit of a patient circuit fluidly coupled to the second conduit member.

8.-9. (canceled)

10. The patient interface device according to claim 1, wherein the first connector portion and the second connector portion is each cylindrical in shape.

11.-15. (canceled)

16. The patient interface device according to claim 1, wherein the first angle and the second angle are each 45 degrees.

17.-20. (canceled)

21. A fluid coupling conduit for a patient interface device, comprising:
a first conduit member, the first conduit member being structured to be fluidly coupled to at least one of a frame and a cushion of the patient interface device and being rotatable with respect to the frame and about a longitudinal axis of the first conduit member when coupled to the frame; and
a second conduit member, the second conduit member being fluidly coupled to first conduit member and being rotatable with respect to the first conduit member and about an axis that is transverse to the longitudinal axis of the first conduit member,
wherein the first conduit member comprises a first connector portion defining a First fluid chamber, and a first mating portion coupled to the first connector portion, the first mating portion defining a second fluid chamber,
wherein the second conduit member comprises a second connector portion defining a third fluid chamber, and a second mating portion coupled to the second connector portion, the second mating portion defining a fourth fluid chamber,
wherein the first mating portion is fluidly coupled to the second mating portion,
wherein the first mating portion includes a first back surface connected to a first angled surface, the first angled surface being disposed at a first angle with respect to a plane defining a flat top of the first connector portion,
wherein the second mating portion includes a second back surface connected to a second angled surface, the second angled surface being disposed at a second angle with respect to a plane defining a flat top of the second connector portion, and
wherein the first angle is the same as the second angle, and wherein the first angle and the second angle are each less than 90 degrees and greater than 30 degrees.

22. (canceled)

23. The fluid coupling conduit according to claim 21, wherein the first conduit member is rotatable with respect to the frame and about the longitudinal axis of the first conduit member over 360 degrees.

24. The fluid coupling conduit according to claim 21, wherein the second conduit member is rotatable with respect to the first conduit member and about the axis over at least 180 degrees.

25. The fluid coupling conduit according to claim 24, wherein the second conduit member is rotatable with respect to the first conduit member and about the axis over greater than 180 degrees.

26. The fluid coupling conduit according to claim 21, wherein the second conduit member is rotatable with respect to the first conduit member and about the axis over less than 180 degrees.

27. The fluid coupling conduit according to claim 21, further comprising a gas delivery conduit of a patient circuit fluidly coupled to the second conduit member.

28.-29. (canceled)

30. The fluid coupling conduit according to claim 21, wherein the first connector portion and the second connector portion is each cylindrical in shape.

31.-35. (canceled)

36. The fluid coupling conduit according to claim 21, wherein the first angle and the second angle are each 45 degrees.

37.-40. (canceled)
